(19) United States
(12) Patent Application Publication
(10) Pub. No.: US 2022/0362157 A1
(43) Pub. Date: Nov. 17, 2022

(54) NEW HYDROCORTISONE HEMISUCCINATE LYOPHILIZATE

(71) Applicant: LABORATOIRE AGUETTANT, Lyon (FR)

(72) Inventors: Fanny REYNAUD, Lyon (FR); Jeff TONNAR, Lyon (FR)

(73) Assignee: LABORATOIRE AGUETTANT, Lyon (FR)

(21) Appl. No.: 17/764,584

(22) PCT Filed: Oct. 16, 2020
(86) PCT No.: PCT/FR2020/051850

§ 371 Date: Mar. 29, 2022

(30) Foreign Application Priority Data
Oct. 18, 2019 (FR) FR1911670

(51) Int. Cl. A61K 9/19; A61K 31/573; A61K 9/14

(52) U.S. Cl. CPC A61K 9/19; A61K 31/573; A61K 9/145; A61K 9/143

(57) ABSTRACT

A lyophilized powder for the preparation of a solution for intravenous or parenteral injection comprising: from 1% to 30% of hydrocortisone hemisuccinate; from 40% to 98% of a bulking agent chosen from mannitol, trehalose, lactose, sucrose, raffinose or sorbitol; from 1% to 30% of a buffering agent; and use thereof for the preparation of a solution for intravenous or parenteral injection useful for the prevention or the treatment of the bronchopulmonary dysplasia in premature infants.

The object of the present invention is a new hydrocortisone hemisuccinate lyophilizate and its use for the preparation of a solution for intravenous or parenteral injection useful for the prevention or the treatment of the bronchopulmonary dysplasia in premature infants.

The bronchopulmonary dysplasia (BPD) is a chronic lung disease frequently found in premature or very premature infants (i.e., term less than 32 weeks of amenorrhea) with low birth weight and requiring mechanical ventilation as part of acute respiratory distress syndrome. It is one of the main complications and one of the main causes of mortality and morbidity in premature or very premature infants.

In very premature infants, the development of the pulmonary alveoli is not complete and the slightest aggression can alter the maturation. This is the case, in particular, during an infection of the amniotic fluid.

In premature infants, it is a risk factor for neonatal mortality or neurological sequelae. The risk of occurrence of respiratory problems during childhood is also increased. The ventilation function can be impaired, even in adulthood.

The corticosteroids administered intravenously reduce the risk of occurrence of a bronchopulmonary dysplasia in premature or very premature infants. However, the low doses to be administered generally require high dilutions of the solutions on the market. This step of preparing the solution before injection has a significant risk of dosing errors or microbial contamination of the injected solution.

To limit these risks, the Directive EMA/CHMP/QWP/1805880/2012 Rev, 2 relating to the pharmaceutical development of drugs for pediatric use issued by the European Medicines Agency (FMA) recommends avoiding these dilutions by developing formulations containing the appropriate concentrations of active ingredient.

The hydrocortisone hemisuccinate, also known as hydrocortisone succinate hydrogen, hydrogen succinate hydrocortisone, or simply hydrocortisone succinate, is a synthetic corticosteroid marketed in many countries around the world as a drug for the treatment of acute adrenal insufficiency, transient adrenal insufficiency of the newborn and congenital adrenal hyperplasia with salt loss syndrome (Debre-Fibiger syndrome).

This active ingredient is subject to rapid hydrolysis in an aqueous medium due to the unstable ester bond between hydrocortisone and succinate, so much so that it is currently marketed in the form of a lyophilized powder (or lyophilizate) containing 100 mg or 500 mg of hydrocortisone with a reconstitution solution.

Nevertheless, the posology defined in the context of the prevention or of the treatment of bronchopulmonary dysplasia in premature or very premature infants being 0.5 mg of hydrocortisone/kg/12 hours for the first 7 days then 0.5 mg of hydrocortisone/kg/24 hours for the next 3 days, it is necessary to dilute 50 times (using a 5% glucose solution) the solution obtained after reconstitution from the lyophilizate containing 100 mg of hydrocortisone and reconstitution solution (2 ml) to administer it.

On the date of the present invention, there is no formulation enabling the intravenous (or parenteral) administration of hydrocortisone to premature or very premature infants in the aforementioned doses and volumes which could be reconstituted by simply adding a reconstitution solution and without subsequent dilution.

There is therefore a recognized need for such a composition. However, the development of such a composition is very complex. Indeed, it is known that lyophilization is a complex method where several parameters must be considered in order to guarantee the conformity of the product. Although this method allows significantly improving the stability of labile products in an aqueous medium, particular attention must be paid to the choice of the formulation. In particular, the parameters such as the pH, the nature and the amount of excipients in the formulation have a very significant impact on the stability of the lyophilizate finally prepared. In addition, contradictory information about the effect of the different excipients on the stabilization of active pharmaceutical ingredients is available, which does not allow determining with certainty a relationship between the structure of a lyophilizate and its stability, all the more so that the combination of excipients may, depending on their nature and their proportion, unexpectedly affect the stability of the lyophilizate. Finally, the aforementioned difficulties are further increased in the case of a lyophilizate containing small (even very small) amounts of active ingredient. Indeed, a low (even a very low) amount of active ingredient in a lyophilizate presupposes the presence of a large amount of bulking agent, and therefore additional difficulties in developing a stable and easily reconstitutable formulation.

Yet, it has been discovered, in a completely unexpected way, that the use of particular bulking agents combined with the addition of some amount of a buffering agent allowed preparing a lyophilizate containing small (even very small) amounts of hydrocortisone hemisuccinate stable over time and easily soluble in a small volume of reconstitution solution in order to prepare a solution ready to be injected intravenously or parenterally into premature or very premature infants.

Thus, the subject of the present invention is a lyophilized powder for the preparation of a solution for intravenous or parenteral injection comprising:

from 1% to 30% of hydrocortisone hernisuccinate;

from 40% to 98% of a bulking agent chosen from mannitol, trehalose, lactose, sucrose, raffinose or sorbitol;

from 1% to 30% of a buffering agent.

The lyophilized powder according to the invention is stable over time when it is kept at 5° C., even when it is kept at room temperature. In addition, it allows the preparation of a solution for intravenous or parenteral injection in premature (even very premature) infants to prevent and treat the bronchopulmonary dysplasia by simple reconstitution, without subsequent dilution.

In the context of the present invention:

“lyophilized powder” or “‘lyophilizate”’ means any powder or dry powder containing less than 8% of water, preferably less than 5% of water, and prepared by any method allowing reaching such a level of humidity, in particular lyophilization;

“hydrocortisone hemisuccinate” refers to 11β,17α,21-trihydroxypregn-4-ene-3,20-dione 21-hemisuccinate, CAS number 2203-97-6 (monohydrate form) or 83784-20-7 (anhydrous form) and chemical structure:

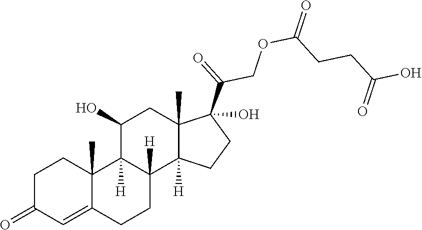

as well as the pharmaceutically acceptable salts hereof (in particular the sodium salt thereof);

“pharmaceutically acceptable salt” of an active ingredient means any addition salt of said active ingredient with a mineral or organic acid by the action of such an acid in an organic or aqueous solvent such as alcohol, ketone, ether or chlorinated solvent, and which is pharmaceutically acceptable; and

“buffering agent” means any compound or combination of compounds allowing maintaining the pH of a solution at a stable value (+/−1, preferably +/−0.5). As an example of a buffering agent, mention may in particular be made of citrate, phosphate, TRIS, histidine and HEPES as well as the associated salts,

In addition, in the context of the present invention, and unless otherwise stated, the proportions expressed in % correspond to mass percentages relative to the total weight of the considered entity.

An object of the present invention is therefore a lyophilized powder containing small (even very small) amounts of hydrocortisone hemisuccinate, a bulking agent and a buffering agent as defined above. Preferentially, an object of the present invention is a lyophilized powder as defined above having the following features, considered alone or in combination: the lyophilized powder contains from 2% to 20% of hydrocortisone hemisuccinate, more preferably from 5% to 15% of hydrocortisone hemisuccinate, and most preferably from 8% to 12% of hydrocortisone hemisuccinate;

the bulking agent is chosen as being mannitol or trehalose, more preferably the bulking agent is chosen as being trehalose; the lyophilized powder contains from 50% to 95% of bulking agent, preferably from 60% to 90% of bulking agent, and most preferably from 75% to 85% of bulking agent;

the buffering agent is chosen as being phosphate or one of the salts thereof, preferably the buffering agent is chosen as being phosphate in the form of sodium salt, most preferably the buffering agent is chosen as being a mixture of monosodium phosphate and disodium phosphate;

the lyophilized powder contains from 2% to 20% of buffering agent, preferably from 5% to 15% of buffering agent, and most preferably from 8% to 12% of buffering agent;

the lyophilized powder also contains one or more compounds allowing the pH to be adjusted, such as sodium hydroxide or orthophosphoric acid.

The lyophilized powder according to the present invention can be prepared according to any method conventionally used by those skilled in the art. As an example of a method for preparing a lyophilized powder as defined above, mention may in particular be made of the method comprising the following steps:

addition at room temperature of the buffering agent at 70% (v/v) to 90% (v/v) of the total water necessary for the preparation of the solution before lyophilization;

addition of the hydrocortisone hemisuccinate;

adjustment of the pH of the solution to a value varying from 7.0 to 8.0, preferably from 7.0 to 7.4;

addition of the bulking agent;

addition of the rest of the necessary water;

filtration of the solution;

lyophilization of the obtained solution.

The lyophilized powder according to the present invention can therefore be used to prepare a solution for intravenous or parenteral injection to premature or very premature infants by simply adding a reconstitution solution.

Thus, the present invention also relates to the use of a lyophilized powder as defined above for the preparation of a solution for intravenous or parenteral injection to premature or very premature infants.

The present invention also relates to a kit for the preparation of a solution for intravenous or parenteral injection to premature or very premature infants comprising; the lyophilized powder as defined previously; and

a reconstitution solution.

Preferably, the kit according to the present invention has the following features, considered alone or in combination:

the kit contains from 1 mg to 50 mg of lyophilized powder, preferably from 5 mg to 30 mg of lyophilized powder, more preferably from 10 mg to 20 mg of lyophilized powder, most preferably 13 mg of lyophilized powder;

the kit contains from 0.1 ml to 10 ml of reconstitution solution, preferably from 0.2 ml to 5 ml of reconstitution solution, more preferably from 0.5 ml to 2 ml of reconstitution solution, most preferably 1 ml of reconstitution solution; and/or

the reconstitution solution is chosen as being water or a mixture of water and an osmotic agent, preferably a mixture of water and glucose.

The solution for intravenous or parenteral injection obtained from the kit according to the present invention can therefore be immediately injected into premature (or very premature) infants for the prevention or the treatment of a bronchopulmonary dysplasia. Thus, the present invention also relates to an injectable solution obtained from the kit as described above for use in the prevention or the treatment of the bronchopulmonary dysplasia in premature or very premature infants.

Finally, the present invention also relates to a method for preventing or treating the bronchopulmonary dysplasia in premature or very premature infants, comprising the administration to said child of a solution prepared from the kit described above.

The present invention is illustrated without limitation by the following examples.

### EXAMPLE 1

### Preparation of a Lyophilized Powder according to the Invention

1.1 Lyophilizate L1

A lyophilized powder L1, the composition of which is reported in the following Table 1, was prepared according to the method described below.

TABLE 1
Lyophilizate L1

| Ingredient | % (w/w) |
|---|---|
| Hydrocortisone hemisuccinate | 10.5% |
| Trehalose (bulking agent) | 79% |
| Sodium phosphate (buffering agent) | 1% |
| Disodium phosphate (buffering agent) | 9.5% |
| Sodium hydroxide (pH adjustment) | qs target pH |
| Orthophosphoric acid (pH adjustment) | qs target pH |

0.23 g of sodium phosphate and 2.41 g of disodium phosphate are solubilized in 800 ml of water for a preparation injectable at room temperature. After stirring and complete dissolution, 2.66 g of hydrocortisone hemisuccinate are added, still with stirring. The pH is adjusted to 7.4 until the active ingredient is completely dissolved. 20 g of trehalose are then added to the solution. All the powders being solubilized, the pH is adjusted to the target, i.e., 7.0 +/−0.1, and the water level is topped up to 1 liter.

The lyophilization is then carried out according to the cycle reported in the following Table 2.

TABLE 2
Lyophilization cycle

| No. | Step | Temperature | | Pressure | | Duration | |
|---|---|---|---|---|---|---|---|
| 1 | Freezing | −45° | C. | Atmospheric | | 1 | hour |
| 2 | Annealing | −25° | C. | Atmospheric | | 5 | hours |
| 3 | Primary dessiccation | −30° | C. | 70 | μbar | 44 | hours |
| 4 | Secondary dessiccation | +30° | C. | 25 | μbar | 10 | hours |

| | | | | | | |
|---|---|---|---|---|---|---|
| 5 | Capping under nitrogen | +5° | C. | 50 | mbar | N/A |

1.2 Lyophilizate

A lyophilized powder L2, the composition of which is reported in Table 3, is prepared according to the method described in Example 1.1 above.

TABLE 3
Lyophilizate L2

| Ingredient | % (w/w) |
|---|---|
| Hydrocortisone hemisuccinate | 17.4% |
| Mannitol (bulking agent) | 65.4% |
| Sodium phosphate (buffering agent) | 1.6% |
| Disodium phosphate (buffering agent) | 15.7% |
| Sodium hydroxide (pH adjustment) | qs target pH |
| Orthophosphoric acid (pH adjustment) | qs target pH |

1.3 Reference

A so-called reference lyophilized powder “L_{ref}”, the composition of which is reported in Table 4, is prepared according to the method described in Example 1.1 above,

TABLE 4
Reference lyophilizate L_{ref.}

| Ingredient | % (w/w) |
|---|---|
| Hydrocortisone hemisuccinate | 2% |
| Glucose (bulking agent) | 97% |
| Disodium phosphate (buffering agent) | 1% |
| Hydrochloric acid | qs target pH |
| Sodium hydroxide | qs target pH |

2. Reconstitution of the Injection Solution

The reconstitution of the injection solution is carried out by adding 1 ml of a water/glucose reconstitution solution (4.5/95,5) to a lyophilizate containing 1 mg of hydrocortisone (L1, L2 or L_{ref}). 1 ml of hydrocortisone solution at 1 mg/ml is thus obtained.

With the lyophilizates L1 and L2, reconstitution is done immediately after a slight stirring of the the vial to homogenize the solution. The reconstitution therefore does not have any difficulty and is carried out as easily as with the lyophilized powders currently on the market which do not contain bulking agent.

The solution can then be withdrawn for injection using a syringe and a needle or a syringe and a transfer device with no further dilution required. Thus, the risk of calculation or handling error that could lead to a dosage error as well as the risk of microbial contamination are greatly reduced compared to the products currently on the market.

On the contrary, with the lyophilizate L_{ref.}, there is a marked tendency to collapse regardless of the storage temperature, which makes the reconstitution more difficult, The reconstitution time is thus about 30 seconds when the lyophilizate is stored at 5° C. and can go up to 150 seconds when the lyophilizate is stored at 25° C. or 40° C. In addition, this marked tendency to collapse reveals an instability over time of the lyophilizate, regardless of the storage temperature.

3. Stability of the Lyophilizates

The lyophilizates L1 and L2 were stored at the temperatures of 5° C., 25° C. and under accelerated stability conditions at 40° C. Analyzes aimed at determining the level of impurities (i.e., free hydrocortisone), and therefore the physico-chemical stability of the formulations, were carried out at T₀, T₀+1 month, T₀+2 months and T₀+3 months.

The results are reported in the following Table 5.

TABLE 5
Evolution of the free hydrocortisone impurity for the
formulations L1 and L2 at T₀, T₀ + 1 month, T₀ +
2 months and T₀ + 3 months at 5° C., 25° C. and 40° C.

Free hydrocortisone ratio (% (w/w))

| Formulation/ | | T₀ + 1 | T₀ + 2 | T₀ + 3 |
|---|---|---|---|---|
| Storage conditions | T₀ | month | months | months |
| L1/5° C. | 0.6 | 0.8 | 0.8 | 0.8 |
| L1/25° C. | 0.6 | 0.9 | 1.0 | 1.1 |
| L1/40° C. | 0.6 | 1.4 | 1.9 | 2.4 |
| L2/5° C. | 0.7 | 0.8 | 0.9 | 1.0 |
| L2/25° C. | 0.7 | 1.8 | 3.0 | 4.0 |
| L2/40° C. | 0.7 | 7.0 | 12.4 | 15.6 |

The tolerated impurity limit ratio is 6.7% (w/w).

It is noted that the lyophilizates L1 and L2 are stable when stored at 5° C. and at 25° C. at T₀+3 months. The lyophilizate L1 remains stable even when stored under accelerated storage conditions at 40° C.

1. A lyophilized powder for the preparation of a solution for intravenous or parenteral injection comprising:
from 1% to 30% of hydrocortisone hemisuccinate;
from 40% to 98% of a bulking agent chosen from mannitol, trehalose, lactose, sucrose, raffinose or sorbitol;
from 1% to 30% of a buffering agent.

2. The lyophilized powder according to claim 1, wherein it contains from 5% to 15% of hydrocortisone hemisuccinate.

3. The lyophilized powder according to claim 1, wherein the bulking agent is chosen as being mannitol or trehalose.

4. The lyophilized powder according to claim 1, wherein it contains from 60% to 90% of bulking agent.

5. The lyophilized powder according to claim 1, wherein the buffering agent is chosen as being citrate, phosphate, TRIS, histidine or HEPES as well as the associated salts.

6. The lyophilized powder according to claim 1, wherein it contains from 5% to 15% of buffering agent.

7. A method of preparing a lyophilized powder according to claim 1 comprising the following steps:
addition at room temperature of the buffering agent at 70% (v/v) to 90% (v/v) of the total water necessary for the preparation of the solution before lyophilization;
addition of the hydrocortisone hemisuccinate;
adjustment of the pH of the solution to a value varying from 7 to 8;
addition of the bulking agent;
addition of the rest of the necessary water;
filtration of the solution;
lyophilization of the obtained solution.

8. A use of a lyophilized powder according to claim 1 for the preparation of a solution for intravenous or parenteral injection in premature or very premature infants.

9. A kit for the preparation of a solution for intravenous or parenteral injection in premature or very premature infants comprising:
the lyophilized powder according to claim 1; and
a reconstitution solution.

10. A solution for intravenous or parenteral injection obtained from the kit according to claim 9 for use in the prevention or the treatment of the bronchopulmonary dysplasia in premature or very premature infants.
